SEQUENCE LISTING (13 sequences)

SEQ ID NO: 1 - protein, 5 aa, synthetic construct
DFYME

SEQ ID NO: 2 - protein, 19 aa, synthetic construct
ASRNKANDYTTEYADSVKG

SEQ ID NO: 3 - protein, 12 aa, synthetic construct
DYYGSSYWYFDV

SEQ ID NO: 4 - protein, 17 aa, synthetic construct
TASESLYSSKHKVHYLA

SEQ ID NO: 5 - protein, 7 aa, synthetic construct
GASNRYI

SEQ ID NO: 6 - protein, 9 aa, synthetic construct
AQFYSYPLT

SEQ ID NO: 7 - protein, 123 aa, synthetic construct
EVKLVESGGGLVQPGGSLRLSCATSGFTFSDFYMEWVRQAPGKRLEWIAASRNKANDYTTEYADSVKGRFIVSRDTSQSILYLQMNALRAEDTAIYYCARDYYGSSYWYFDVWGAGTTVTVSS

SEQ ID NO: 8 - protein, 113 aa, synthetic construct
DIVMTQSPSSLSVSAGKKVTISCTASESLYSSKHKVHYLAWYQKKPEQSPKLLIYGASNRYIGVPDRFTGSGSGTDFTLTISSVQVEDLTHYYCAQFYSYPLTFGAGTKLEIK

SEQ ID NO: 9 - protein, 15 aa, synthetic construct
GGGGSGGGGSGGGGS

SEQ ID NO: 10 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - protein, 725 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 13 - DNA, 133 bp, synthetic construct
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

(19) United States
(12) Patent Application Publication
(10) Pub. No.: US 2026/0297217 A1
(43) Pub. Date: Oct. 1, 2026

(54) METHODS OF TREATING OXIDIZED PHOSPHATIDYLCHOLINE-ASSOCIATED DISEASES

(71) Applicant: VECTORY THERAPEUTICS B.V., Amsterdam (NL)

(72) Inventors: Pavlina KONSTANTINOVA, Amsterdam (NL); Sander VAN DEVENTER, Amsterdam (NL); Andreia GOMES-DUARTE, Amsterdam (NL); Svitlana PASTEUNING-VUHMAN, Amsterdam (NL); Wouter POS, Amsterdam (NL)

(21) Appl. No.: 19/478,622

(22) PCT Filed: Apr. 29, 2024
(86) PCT No.: PCT/IB2024/000205

§ 371 Date: Oct. 24, 2025

Related U.S. Application Data
(60) Provisional application No. 63/498,932, filed on Apr. 28, 2023.

(51) Int. Cl. C07K 16/44; A61K 39/00; A61P 25/28; C07K 14/005; C12N 15/86

(52) U.S. Cl. CPC C07K 16/44; A61P 25/28; C07K 14/005; C12N 15/86; A61K 2039/505; C07K 2317/24; C07K 2317/565; C07K 2317/622; C07K 2317/76; C12N 2750/14122; C12N 2750/14143; C12N 2750/14145

(57) ABSTRACT

Provided herein are methods of treating diseases and disorders related to TDP-43 aggregation (e.g., ALS) with an antibody that specifically binds to OxPC or a polynucleotide encoding an antibody that specifically binds to OxPC.

### RELATED APPLICATION

This application claims benefit to U.S. Provisional Application No. 63/498,932, filed on Apr. 28, 2023, the content of which is incorporated by reference herein in its entirety.

### SEQUENCE LISTING

The content of the electronically submitted Sequence Listing XML (Name: 209266_SL.xml; Size: 14,693 bytes; Created on Apr. 17, 2024) is incorporated by reference herein in its entirety.

### BACKGROUND

Amyotrophic lateral sclerosis (ALS) is a neurodegenerative disease that causes progressive loss of motor neurons, eventually leading to paralysis and premature death. It is a multifactorial disease characterized by protein aggregation, neuroinflammation, mitochondrial dysfunction, axonal defects, and neuromuscular junction (NMJ) damage. Most patients diagnosed with ALS will die within three to five years, often from respiratory failure. Currently, there is no cure for ALS and most treatments rely on symptom management.

Misfolded TAR DNA-binding protein-43 (TDP-43) has been associated with the pathology of 97% of sporadic ALS, as well as genetic forms of ALS. In ALS, misfolded TDP-43 interferes with the translation of mitochondrial proteins at the neuromuscular junction (NMJ) leading to mitochondrial dysfunction. Besides TDP-43 pathology, the main metabolic abnormality in ALS motor neurons is a dramatic increase of glycerophospholipids triggering the formation of neurotoxic oxidized phosphatidylcholines (OxPCs), a type of oxidized phospholipid (OxPL). OxPCs have been implicated in multiple diseases, such as multiple sclerosis and ALS, and thus, targeting OxPCs is a potential new therapeutic approach for treating these diseases.

Accordingly, there is a need in the art for ALS treatments that effectively reduce or eliminate symptoms.

### SUMMARY

Provided herein are methods of inhibiting OxPL-induced neurotoxicity, inhibiting TDP-43 aggregate-induced neurotoxicity, preventing or reducing TDP-43 aggregates, and/or treating a disease or disorder associated with an increased level of oxidized phosphatidylcholine (OxPC) in a subject in need thereof by administering to the subject an antibody or antigen-binding fragment thereof that specifically binds to OxPC; or a polynucleotide encoding an antibody or antigen-binding fragment thereof that specifically binds to OxPC. The methods disclosed herein are particularly advantageous because they prevent and/or reduce TDP-43 aggregates in subjects who have a condition associated with TDP-43 aggregates (e.g., ALS) and prevent OxPC induced neurotoxicity.

In an aspect, provided herein is a method of inhibiting oxidized phospholipid (OxPL)-induced neurotoxicity in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an aspect, provided herein is a method of inhibiting TDP-43 aggregate-induced neurotoxicity in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an aspect, provided herein is a method of preventing or reducing TDP-43 aggregates in a subject suffering from a condition associated with TDP-43 aggregate formation, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an embodiment, the subject has amyotrophic lateral sclerosis (ALS), Alzheimer's disease, motor neuron disease, Parkinson's disease, or frontotemporal lobar degeneration. In an embodiment, the subject has sporadic ALS (sALS).

In an aspect, provided herein is a method of treating a disease or disorder associated with an increased level of oxidized phosphatidylcholine (OxPC) in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an embodiment, the disease or disorder is amyotrophic lateral sclerosis (ALS), Alzheimer's disease, motor neuron disease, Parkinson's disease, or frontotemporal lobar degeneration. In an embodiment, the disease or disorder is sporadic ALS (sALS).

In an embodiment, the antibody or antigen-binding fragment comprises a heavy chain variable region comprising complementarity determining regions CDRH1, CDRH2, and CDRH3 of the heavy chain variable region amino acid sequence set forth in SEQ ID NO: 7, and a light chain variable region comprising complementarity determining regions CDRL1, CDRL2, and CDRL3 of the light chain variable region amino acid sequence set forth in SEQ ID NO: 8.

In an embodiment, the antibody or antigen-binding fragment comprises a CDRH1, CDRH2, CDRH3, CDRL1, CDRL2, and CDRL3 comprising the amino acid sequences set forth in SEQ ID NOs: 1, 2, 3, 4, 5, and 6, respectively.

In an embodiment, the antibody or antigen-binding fragment thereof comprises a heavy chain variable region comprising an amino acid sequence with at least 80% identity to SEQ ID NO: 7. In an embodiment, the antibody or antigen-binding fragment thereof comprises a light chain variable region comprising an amino acid sequence with at least 80% identity to SEQ ID NO: 8.

In an embodiment, the antibody or antigen-binding fragment thereof comprises a heavy chain variable region comprising an amino acid sequence with at least 80% identity to the amino acid sequence set forth in SEQ ID NO: 7, and a light chain variable region comprising an amino acid sequence with at least 80% identity to the amino acid sequence set forth in SEQ ID NO: 8.

In an embodiment, the antibody or antigen-binding fragment thereof is a humanized antibody.

In an embodiment, the antibody or antigen-binding fragment thereof is a single chain variable fragment (scFv).

In an embodiment, the scFv comprises a peptide linker between the heavy chain variable region and the light chain variable region. In an embodiment, the peptide linker comprises the amino acid sequence of GGGGSGGGGSGGGGS (SEQ ID NO: 9).

In an embodiment, the polynucleotide is comprised within a vector. In an embodiment, the vector is a viral vector.

In an embodiment, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In an embodiment, the vector is an AAV vector comprised within a recombinant AAV (rAAV), wherein the rAAV comprises an AAV capsid comprising an AAV capsid protein; and a rAAV genome.

In an embodiment, the capsid protein is a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

In an embodiment, the capsid protein comprises an amino acid sequence that has at least 95% identity to amino acids 193-725 of SEQ ID NO: 11. In an embodiment, the capsid protein comprises an amino acid sequence that has at least 99% identity to amino acids 193-725 of SEQ ID NO: 11. In an embodiment, the capsid protein comprises the amino acid sequence of amino acids 193-725 of SEQ ID NO: 11.

In an embodiment, the capsid protein comprises an amino acid sequence that has at least 95% identity to amino acids 138-725 of SEQ ID NO: 11. In an embodiment, the capsid protein comprises an amino acid sequence that has at least 99% identity to amino acids 138-725 of SEQ ID NO: 11. In an embodiment, the capsid protein comprises the amino acid sequence of amino acids 138-725 of SEQ ID NO: 11.

In an embodiment, the capsid protein comprises an amino acid sequence that has at least 95% identity to SEQ ID NO: 10 or 11. In an embodiment, the capsid protein comprises an amino acid sequence that has at least 99% identity to SEQ ID NO: 10 or 11. In an embodiment, the capsid protein comprises the amino acid of SEQ ID NO: 10 or 11.

In an embodiment, the rAAV genome further comprises a CBh promoter comprising the nucleic acid sequence set forth in SEQ ID NO: 12.

In an embodiment, the rAAV genome further comprises an SV40 polyA tail comprising the nucleic acid sequence set forth in SEQ ID NO: 13.

In an embodiment, the rAAV is administered to the subject intravenously, intraperitoneally, subcutaneously, intramuscularly, intrathecally, or intradermally.

In an embodiment, the method neutralizes OxPC activity in the subject.

In an embodiment, the method prevents or reduces TDP-43 aggregates.

In an embodiment, the method reduces the expression of one or more ALS related gene. In an embodiment, the one or more ALS related gene is apoE, COL4A1, CTSS, DAB2, TIMP1, or any combination thereof.

In an embodiment, the method reduces the expression of one or more ALS related gene. In an embodiment, the one or more ALS related gene is C9orf72, GRM3, SYP, GRIN2B, CHRNA7, MYD88, ITPR2, GRN, VEGFA, FKBP5, or any combination thereof.

In an embodiment, the method neutralizes OxPC mediated neurotoxicity. In an embodiment, the method increases electrical firing of a neuron. In an embodiment, the method increases neurite outgrowth on a neuron.

In an embodiment, the vector targets a cortical neuron, a spinal neuron, and/or an astrocyte.

In an embodiment, the subject is a human subject.

In an aspect, provided herein is an antibody or antigen-binding fragment thereof that specifically binds to OxPC or a nucleic acid sequence encoding an antibody or antigen-binding fragment thereof that specifically binds to OxPC for use in inhibiting TDP-43 aggregate-induced neurotoxicity in a subject in need thereof, wherein the treatment is performed according to any one of the methods disclosed herein.

In an aspect, provided herein is an antibody or antigen-binding fragment thereof that specifically binds to OxPC or a nucleic acid sequence encoding an antibody or antigen-binding fragment thereof that specifically binds to OxPC for use in inhibiting oxidized phospholipid (OxPL)-induced neurotoxicity in a subject in need thereof, wherein the treatment is performed according to any one of the methods disclosed herein.

In an aspect, provided herein is an antibody or antigen-binding fragment thereof that specifically binds to OxPC or a nucleic acid sequence encoding an antibody or antigen-binding fragment thereof that specifically binds to OxPC for use in preventing or reducing TDP-43 aggregates in a subject in need thereof, wherein the treatment is performed according to any one of the methods disclosed herein.

In an aspect, provided herein is an antibody or antigen-binding fragment thereof that specifically binds to OxPC or a nucleic acid sequence encoding an antibody or antigen-binding fragment thereof that specifically binds to OxPC for use in the manufacture of a medicament for inhibiting TDP-43 aggregate-induced neurotoxicity in a subject in need thereof, wherein the treatment is performed according to any one of the methods disclosed herein.

In an aspect, provided herein is an antibody or antigen-binding fragment thereof that specifically binds to OxPC or a nucleic acid sequence encoding an antibody or antigen-binding fragment thereof that specifically binds to OxPC for use in the manufacture of a medicament for inhibiting oxidized phospholipid (OxPL)-induced neurotoxicity in a subject in need thereof, wherein the treatment is performed according to any one of the methods disclosed herein.

In an aspect, provided herein is an antibody or antigen-binding fragment thereof that specifically binds to OxPC or a nucleic acid sequence encoding an antibody or antigen-binding fragment thereof that specifically binds to OxPC for use in the manufacture of a medicament for preventing or reducing TDP-43 aggregates in a subject in need thereof, wherein the treatment is performed according to any one of the methods disclosed herein.

In an aspect, provided herein is a use of an antibody or antigen-binding fragment thereof that specifically binds to OxPC or a nucleic acid sequence encoding an antibody or antigen-binding fragment thereof that specifically binds to OxPC for inhibiting TDP-43 aggregate-induced neurotoxicity in a subject in need thereof, wherein the treatment is performed according to any one of the methods disclosed herein.

In an aspect, provided herein is a use of an antibody or antigen-binding fragment thereof that specifically binds to OxPC or a nucleic acid sequence encoding an antibody or antigen-binding fragment thereof that specifically binds to OxPC for inhibiting oxidized phospholipid (OxPL)-induced neurotoxicity in a subject in need thereof, wherein the treatment is performed according to any one of the methods disclosed herein.

In an aspect, provided herein is a use of an antibody or antigen-binding fragment thereof that specifically binds to OxPC or a nucleic acid sequence encoding an antibody or antigen-binding fragment thereof that specifically binds to OxPC for preventing or reducing TDP-43 aggregates in a subject in need thereof, wherein the treatment is performed according to any one of the methods disclosed herein.

### BRIEF DESCRIPTION OF THE DRAWINGS

FIG. 1 is a graph showing OxPC expression in SOD1^{G93A} ALS motor neurons and healthy motor neurons (% OxPC+ cells). Values are expressed as means (Healthy=1.000; SOD1^{G93A}=1.239)±SEM and were normalized to Healthy; ***p=0.0008. Unpaired two-tailed t-test; three independent experiments, N=2-4 replicates each.

FIG. 2A is a graph showing the effect of OxPC (PONPC) treatment of healthy motor neurons on neuronal outgrowth. Values are expressed as means±SEM and were normalized to 25 μM PSPC condition. Two-way ANOVA (F statistic=25.67; p=0.0368) with Tukey post hoc test (p<0.05); n=3 independent experiments, 1-4 replicates. FIG. 2B is a graph showing elevated apoE profile (% positive cells and average intensity) in SOD1^{G93A} ALS motor neurons compared to healthy motor neurons. Values are expressed as means±SEM and were normalized to Healthy; *p=0.0241, ****p<0.0001. Unpaired two-tailed t-test; two independent experiments, n=2 replicates each. FIG. 2C is graph showing increased apoE profile in SOD1^{G93A} ALS motor neurons at 24-hour OxPC treatment+6-hour washout or 24-hour OxPC treatment+24-hour washout. Scale bar=20 μM (apoE, FITC). FIG. 2D is a graph showing increased TDP-43 aggregation in healthy motor neurons treated with OxPC or TDP-43^{M337V} ALS motor neurons treated with OxPC. FIG. 2E is a graph showing pTDP-43 levels in healthy motor neurons treated with OxPC.

FIG. 3A is a graph showing that AAV-mE06 exhibits differential tropism among human derived neurons and astrocytes (GFP (FITC), green fluorescent protein). Scale bar=300 μM. FIG. 3B shows mE06-scFv transcript expression following AAV transduction at different multiplicities of infection (MOIs) in motor neurons and astrocytes. Scatter plot displays gene expression mean, normalized to Hprt1 and relative to non-transduced condition (background). N=3 (Hprt1, Hypoxanthine Phosphoribosyltransferase 1). FIG. 3C shows mE06-scFv protein expression following AAV transduction of motor neurons and astrocytes. Both cell types were transduced with AAV-mE06-scFv at different MOIs. Scatter plot displays gene expression mean. Maximum signal (OD450) using a 4× dilution was used to estimate scFv concentration. N=3.

FIG. 4A is a graph showing TDP-43 aggregation in healthy motor neurons and TDP-43^{M337V} ALS motor neurons transduced with AAV5.2-mE06 and treated with OxPC. FIG. 4B is a graph showing the number of axons in SOD1^{G93A} ALS motor neurons transduced with AAV5.2-mE06 and treated with OxPC. Values are expressed as means±SEM and were normalized to 25 μM PSPC condition. The number of axons was counted on the distal compartment according to ImageJ plugin and Tuj-1 used as a neuronal marker. N=2 experiments. FIG. 4C is a graph showing electrical bursts in healthy motor neurons and TDP-43^{M337V} ALS motor neurons transduced with AAV5.2-mE06 and treated with OxPC. Values are expressed as means±SEM and were normalized to non-transduced neurons treated with OxPC (PONPC). N=4 experiments.

FIG. 5A is a graph showing motor function deficit score in the sALS-CSF mice model transduced with AAV5.2-mE06. FIG. 5B is a graph showing grip strength normalized to baseline in the sALS-CSF mouse model transduced with AAV5.2-mE06. FIG. 5C is a graph showing the number of choline acetyltransferase positive (ChAT⁺) motor neurons in the sALS-CSF mouse model transduced with AAV5.2-mE06 and treated with OxPC. Motor deficit scores, normalized grip strength scores, and immunostaining intensities were each analyzed using a one-way ANOVA, with Bonferroni post hoc analyses. N=3-6 mice per experiment.

FIG. 6A-FIG. 6G are a series of graphs showing the concentration in ng/ml of various species of OxPC in plasma from wild-type, PBS-treated SOD1^{G93A} transgenic mice, and SOD1^{G93A} transgenic mice treated with AAV5.2-mE06. Differences between OxPC concentrations were compared using an unpaired t-test with Welch's correction (non-equal standard deviations).

### DETAILED DESCRIPTION

Provided herein are methods of inhibiting oxidized phospholipid (OxPL)-induced neurotoxicity, inhibiting TDP-43 aggregate-induced neurotoxicity, preventing or reducing TDP-43 aggregates, and/or treating a disease or disorder associated with an increased level of oxidized phosphatidylcholine (OxPC) in a subject in need thereof by administering to the subject an antibody or antigen-binding fragment thereof that specifically binds to OxPC; or a polynucleotide encoding an antibody or antigen-binding fragment thereof that specifically binds to OxPC. The methods disclosed herein are particularly advantageous because they prevent and/or reduce TDP-43 aggregates in subjects who have a condition associated with TDP-43 aggregates (e.g., ALS) and prevent OxPC induced neurotoxicity.

### I. Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, F(ab′)2 fragments, disulfide-linked Fvs (sdFv), anti-idiotypic (anti-Id) antibodies (including, e.g., anti-anti-Id antibodies), variable domain of new antigen receptors (VNARs), antigen binding (Fab) fragments, monobodies, DARPins, VHH antibodies, and antigen-binding fragments of any of the above. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of proteins of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In an embodiment, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In an embodiment, the term “CDR” is a CDR as defined by Kabat et al., J. Biol. Chem. 252, 6609-6616 (1977) and Kabat et al., Sequences of proteins of immunological interest. (1991). In an embodiment, heavy chain CDRs and light chain CDRs of an antibody are defined using different conventions. In an embodiment, heavy chain CDRs and/or light chain CDRs are defined by performing structural analysis of an antibody and identifying residues in the variable region(s) predicted to make contact with an epitope region of a target molecule. CDRH1, CDRH2 and CDRH3 denote the heavy chain CDRs, and CDRL1, CDRL2 and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FRs), Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of proteins of immunological interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fe receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an antibody), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. When a sequence is described herein as being a certain percentage identical to a reference sequence, the percentage identity to the reference sequence is determined across the full length of the reference sequence.

As used herein, the term “subject” includes any human or non-human animal. In an embodiment, the subject is a non-human mammal. In an embodiment, the subject is a human.

As used herein, the term “effective amount” in the context of the administration of an AAV to a subject refers to the amount of the AAV that achieves a desired prophylactic or therapeutic effect.

As used herein, the term “about” or “approximately” when referring to a measurable value, such as a dosage, encompasses variations of ±20% or ±10%, ±5%, ±1%, or ±0.1% of a given value or range, as are appropriate to perform the methods disclosed herein.

### II. Anti-Oxidized Phosphatidylcholine (OxPC) Antibodies

Antibodies that specifically bind to OxPC (i.e., anti-OxPC antibodies) that are useful in the methods described herein include but are not limited to those listed below.

In an embodiment, the antibody comprises: a heavy chain variable region (VH) comprising the CDRH1, CDRH2, and CDRH3 amino acid sequences of the VH amino acid sequence set forth in SEQ ID NO: 7. In an embodiment, the antibody comprises a heavy chain variable region (VH) comprising the CDRH1, CDRH2, and CDRH3 amino acid sequences of the VH amino acid sequence set forth in SEQ ID NO: 7 and a light chain variable region (VL) comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences set forth in SEQ ID NO: 1, 2, and 3, respectively. In an embodiment, the antibody comprises the CDRL1, CDRL2, and CDRL3 amino acid sequences set forth in SEQ ID NO: 4, 5, and 6, respectively. In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises a VH comprising the amino acid sequence set forth in SEQ ID NO: 7. In an embodiment, the antibody comprises a VL comprising the amino acid sequence set forth in SEQ ID NO: 8. In an embodiment, the antibody comprises: a VH comprising the amino acid sequence which is at least 80%, 85%, 90%, 95%, 96%, 97%, 98%, 99%, or 100% identical to the amino acid sequence set forth in SEQ ID NO: 7; and a VL comprising an amino acid sequence which is at least 80%, 85%, 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8.

In an embodiment, the antibody comprises a heavy chain constant region selected from the group consisting of human IgG₁, IgG₂, IgG₃, IgG₄, IgA₁, and IgA₂. In an embodiment, the heavy chain constant region is IgG₁. In an embodiment, the heavy chain constant region is IgG₂. In an embodiment, the antibody comprises a human kappa light chain constant region or a human lambda light chain constant region.

In an embodiment the antibody is an scFv. In an embodiment, the antibody is mouse E06 (mE06), the amino acid sequences of which are provided in Table 1 below.

TABLE 1
Amino acid sequences of mouse E06

| | SEQ | |
|---|---|---|
| | ID | |
| Description | NO: | Amino acid sequence |
| mE06 CDRH1 | 1 | DFYME |
| mE06 CDRH2 | 2 | ASRNKANDYTTEYADSVKG |
| mE06 CDRH3 | 3 | DYYGSSYWYFDV |
| mE06 CDRL1 | 4 | TASESLYSSKHKVHYLA |
| mE06 CDRL2 | 5 | GASNRYI |
| mE06 CDRL3 | 6 | AQFYSYPLT |
| mE06 VH | 7 | EVKLVESGGGLVQPGGSLRLSCATSGFTFSDF |
| | | YMEWVRQAPGKRLEWIAASRNKANDYTTEYAD |
| | | SVKGRFIVSRDTSQSILYLQMNALRAEDTAIY |
| | | YCARDYYGSSYWYFDVWGAGTTVTVSS |
| mE06 VL | 8 | DIVMTQSPSSLSVSAGKKVTISCTASESLYSS |
| | | KHKVHYLAWYQKKPEQSPKLLIYGASNRYIGV |
| | | PDRFTGSGSGTDFTLTISSVQVEDLTHYYCAQ |
| | | FYSYPLTFGAGTKLEIK |

In an embodiment, the scFv comprises a peptide linker between the heavy chain variable region and the light chain variable region. In an embodiment, the peptide linker comprises the amino acid sequence of GGGGSGGGGSGGGGS (SEQ ID NO: 9).

### III. Polynucleotides and Vectors

Polynucleotides and vectors that encode an antibody or antigen binding fragment thereof that specifically binds to OxPC (i.e., anti-OxPC antibodies) that are useful in the methods described herein include but are not limited to those listed below.

In an aspect, provided herein is a method of inhibiting oxidized phospholipid (OxPL)-induced neurotoxicity in a subject in need thereof, the method comprising administering to the subject a therapeutically effective amount of a nucleic acid sequence encoding an antibody or antigen-binding fragment thereof that specifically binds to OxPC.

In an aspect, provided herein is a method of inhibiting TDP-43 aggregate-induced neurotoxicity in a subject in need thereof, the method comprising administering to the subject a therapeutically effective amount of: (a) an antibody or antigen-binding fragment thereof that specifically binds to OxPC; or (b) polynucleotide encoding an antibody or antigen-binding fragment thereof that specifically binds to OxPC.

In an aspect, provided herein is a method of preventing or reducing TDP-43 aggregates in a subject suffering from a condition associated with TDP-43 aggregate formation, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an aspect, provided herein is a method of treating a disease or disorder associated with an increased level of oxidized phosphatidylcholine (OxPC) in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an embodiment, the polynucleotide is comprised within a vector.

In an embodiment, the vector is a non-viral vector. Exemplary non-viral vectors include, but are not limited to, plasmid DNA, transposons, episomal plasmids, minicircles, ministrings, and oligonucleotides (e.g., mRNA, naked DNA). In an embodiment, the non-viral vector is a transposon-based vector. In an embodiment, the non-viral vector is a PiggyBac-based vector, or a Sleeping Beauty-based vector.

In an embodiment, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV1, AAV2, AAV3, AAV4, AAV5, AAV6, AAV7, AAV8, AAV9), retrovirus vectors (e.g., MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector, Newcastle disease virus vectors, poxvirus vectors, and picornavirus vectors (e.g., Coxsackievirus). In an embodiment, the viral vector is selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

In an embodiment, the vector targets a cortical neuron, a spinal neuron, and/or an astrocyte.

In an embodiment, the vector is an AAV vector comprised within a recombinant AAV (rAAV), wherein the rAAV comprises an AAV capsid comprising an AAV capsid protein; and a rAAV genome.

In an embodiment, the capsid protein is a clade A, clade B, clade C, clade D, clade E, clade F, clade G, clade H, clade I, AAVgo.1, AAV3, AAV4, AAV10, AAV11, AAV12, rh.32, rb32.33, rh.33, rh.34, BAAV, or AAV5 capsid protein, or an engineered variant thereof.

In an embodiment, the capsid protein is an engineered variant capsid protein comprising amino acid sequences from at least two different AAV capsid proteins. In an embodiment, the capsid protein comprises amino acid sequences from an AAV2 capsid protein and an AAV5 capsid protein. In an embodiment, the capsid protein comprises a VP1 amino acid sequence from AAV2 and a VP2 and VP3 amino acid sequence from AAV5.

In an embodiment, the capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to amino acids 193-725 of SEQ ID NO: 11. In an embodiment, the capsid protein comprises an amino acid sequence that has at least 99% identity to amino acids 193-725 of SEQ ID NO: 11. In an embodiment, the capsid protein comprises the amino acid sequence of amino acids 193-725 of SEQ ID NO: 11.

In an embodiment, the capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to amino acids 138-725 of SEQ ID NO: 11. In an embodiment, the capsid protein comprises an amino acid sequence that has at least 99% identity to amino acids 138-725 of SEQ ID NO: 11. In an embodiment, the capsid protein comprises the amino acid sequence of amino acids 138-725 of SEQ ID NO: 11.

In an embodiment, the capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to SEQ ID NO: 10 or 11. In an embodiment, the capsid protein comprises an amino acid sequence that has at least 99% identity to SEQ ID NO: 10 or 11. In an embodiment, the capsid protein comprises the amino acid of SEQ ID NO: 10 or 11.

In an embodiment, the rAAV genome further comprises a CBh promoter comprising a nucleic acid sequence that has at least 80%, 81%, 82%, 83%, 84%, 85%, 86%, 87%, 88%, 89%, 90%, 91%, 92%, 93%, 94%, 95%, 96%, 97%, or 98% identity to SEQ ID NO: 12. In an embodiment, the rAAV genome further comprises a CBh promoter comprising the nucleic acid sequence set forth in SEQ ID NO: 12.

| | | |
|---|---|---|
| CBh | SEQ ID | CGTTACATAACTTACGGTAAATGGCCCGCCTG |
| promoter | NO: 12 | GCTGACCGCCCAACGACCCCCGCCCATTGACG |
| | | TCAATAGTAACGCCAATAGGGACTTTCCATTG |
| | | ACGTCAATGGGTGGAGTATTTACGGTAAACTG |
| | | CCCACTTGGCAGTACATCAAGTGTATCATATG |
| | | CCAAGTACGCCCCCTATTGACGTCAATGACGG |
| | | TAAATGGCCCGCCTGGCATTGTGCCCAGTACA |
| | | TGACCTTATGGGACTTTCCTACTTGGCAGTAC |
| | | ATCTACGTATTAGTCATCGCTATTACCATGGT |
| | | CGAGGTGAGCCCCACGTTCTGCTTCACTCTCC |
| | | CCATCTCCCCCCCCTCCCCACCCCCAATTTTG |
| | | TATTTATTTATTTTTTAATTATTTTGTGCAGC |
| | | GATGGGGGCGGGGGGGGGGGGGGGGCGCGCGC |
| | | CAGGCGGGGGGGGCGGGGCGAGGGGGGGGGCG |
| | | GGGCGAGGCGGAGAGGTGCGGCGGCAGCCAAT |
| | | CAGAGCGGCGCGCTCCGAAAGTTTCCTTTTAT |
| | | GGCGAGGCGGCGGCGGCGGCGGCCCTATAAAA |
| | | AGCGAAGCGCGCGGCGGGCGGGAGTCGCTGCG |
| | | CGCTGCCTTCGCCCCGTGCCCCGCTCCGCCGC |
| | | CGCCTCGCGCCGCCCGCCCCGGCTCTGACTGA |
| | | CCGCGTTACTCCCACAGGTGAGCGGGCGGGAC |
| | | GGCCCTTCTCCTCCGGGCTGTAATTAGCTGAG |
| | | CAAGAGGTAAGGGTTTAAGGGATGGTTGGTTG |
| | | GTGGGGTATTAATGTTTAATTACCTGGAGCAC |
| | | CTGCCTGAAATCACTTTTTTTCAGGTTGG |

In an embodiment, the rAAV genome further comprises an SV40 polyA tail comprising a nucleic acid sequence that has at least 80%, 81%, 82%, 83%, 84%, 85%, 86%, 87%, 88%, 89%, 90%, 91%, 92%, 93%, 94%, 95%, 96%, 97%, or 98% identity to SEQ ID NO: 13. In an embodiment, the rAAV genome further comprises an SV40 polyA tail comprising the nucleic acid sequence set forth in SEQ ID NO: 13.

| | | |
|---|---|---|
| SV40 | SEQ ID | TGCTTTATTTGTGAAATTTGTGATGCTATTGCTTT |
| polyA | NO: 13 | ATTTGTAACCATTATAAGCTGCAATAAACAAGTTA |
| | | ACAACAACAATTGCATTCATTTTATGTTTCAGGTT |
| | | CAGGGGGAGGTGTGGGAGGTTTTTTAAA |

In an embodiment, the capsid protein comprises a peptide that targets a cortical neuron, a spinal neuron, and/or an astrocyte.

In an embodiment, the rAAV comprises an AAV capsid comprising an AAV capsid protein, and a rAAV genome comprising a nucleic acid encoding an antibody or antigen binding fragment thereof that specifically binds to OxPC.

In an embodiment, the rAAV genome further comprises a 5′ inverted terminal repeat (5′ ITR) nucleotide sequence, and a 3′ inverted terminal repeat (3′ ITR) nucleotide sequence. ITR sequences from any AAV serotype or variant thereof can be used in the rAAV genomes disclosed herein. The 5′ and 3′ ITR can be from an AAV of the same serotype or from AAVs of different serotypes.

Exemplary capsid protein sequences are disclosed in Table 2 below.

TABLE 2
Exemplary capsid protein sequences

| | SEQ | |
|---|---|---|
| Capsid | ID | |
| protein | NO: | Amino acid sequence |
| AAV5 | 10 | MSFVDHPPDWLEEVGEGLREFLGLEAGPPKPKPNQQ |
| | | HQDQARGLVLPGYNYLGPGNGLDRGEPVNRADEVAR |
| | | EHDISYNEQLEAGDNPYLKYNHADAEFQEKLADDTS |
| | | FGGNLGKAVFQAKKRVLEPFGLVEEGAKTAPTGKRI |
| | | DDHFPKRKKARTEEDSKPSTSSDAEAGPSGSQQLQI |
| | | PAQPASSLGADTMSAGGGGPLGDNNQGADGVGNASG |
| | | DWHCDSTWMGDRVVTKSTRTWVLPSYNNHQYREIKS |
| | | GSVDGSNANAYFGYSTPWGYFDFNRFHSHWSPRDWQ |
| | | RLINNYWGFRPRSLRVKIFNIQVKEVTVQDSTTTIA |
| | | NNLTSTVQVFTDDDYQLPYVVGNGTEGCLPAFPPQV |
| | | FTLPQYGYATLNRDNTENPTERSSFFCLEYFPSKML |
| | | RTGNNFEFTYNFEEVPFHSSFAPSQNLFKLANPLVD |
| | | QYLYRFVSTNNTGGVQFNKNLAGRYANTYKNWFPGP |
| | | MGRTQGWNLGSGVNRASVSAFATTNRMELEGASYQV |
| | | PPQPNGMTNNLQGSNTYALENTMIFNSQPANPGTTA |
| | | TYLEGNMLITSESETQPVNRVAYNVGGQMATNNQSS |
| | | TTAPATGTYNLQEIVPGSVWMERDVYLQGPIWAKIP |
| | | ETGAHFHPSPAMGGFGLKHPPPMMLIKNTPVPGNIT |
| | | SFSDVPVSSFITQYSTGQVTVEMEWELKKENSKRWN |
| | | PEIQYTNNYNDPQFVDFAPDSTGEYRTTRPIGTRYL |
| | | TRPL |
| AAV5.2 | 11 | MAADGYLPDWLEDTLSEGIRQWWKLKPGPPPPKPAE |
| | | RHKDDSRGLVLPGYKYLGPFNGLDKGEPVNEADAAA |
| | | LEHDKAYDRQLDSGDNPYLKYNHADAEFQERLKEDT |
| | | SFGGNLGRAVFQAKKRVLEPLGLVEEPVKTAPTGKR |
| | | IDDHFPKRKKARTEEDSKPSTSSDAEAGPSGSQQLQ |
| | | IPAQPASSLGADTMSAGGGGPLGDNNOGADGVGNAS |
| | | GDWHCDSTWMGDRVVTKSTRTWVLPSYNNHQYREIK |
| | | SGSVDGSNANAYFGYSTPWGYFDFNRFHSHWSPRDW |
| | | QRLINNYWGFRPRSLRVKIFNIQVKEVTVQDSTTTI |
| | | ANNLTSTVQVFTDDDYQLPYVVGNGTEGCLPAFPPQ |
| | | VFTLPQYGYATLNRDNTENPTERSSFFCLEYFPSKM |
| | | LRTGNNFEFTYNFEEVPFHSSFAPSQNLFKLANPLV |
| | | DQYLYRFVSTNNTGGVQFNKNLAGRYANTYKNWFPG |
| | | PMGRTQGWNLGSGVNRASVSAFATTNRMELEGASYQ |
| | | VPPQPNGMTNNLQGSNTYALENTMIFNSQPANPGTT |
| | | ATYLEGNMLITSESETQPVNRVAYNVGGQMATNNQS |
| | | STTAPATGTYNLQEIVPGSVWMERDVYLOGPIWAKI |
| | | PETGAHFHPSPAMGGFGLKHPPPMMLIKNTPVPGNI |
| | | TSFSDVPVSSFITQYSTGQVTVEMEWELKKENSKRW |
| | | NPEIQYTNNYNDPQFVDFAPDSTGEYRTTRPIGTRY |
| | | LTRPL |

### IV. Methods of Treating

In an aspect, provided herein is a method of inhibiting oxidized phospholipid (OxPL)-induced neurotoxicity in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an aspect, provided herein is a method of inhibiting TDP-43 aggregate-induced neurotoxicity in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an aspect, provided herein is a method of preventing or reducing TDP-43 aggregates in a subject suffering from a condition associated with TDP-43 aggregate formation, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an embodiment, the subject has amyotrophic lateral sclerosis (ALS), Alzheimer's disease, motor neuron disease, Parkinson's disease, or frontotemporal lobar degeneration. In an embodiment, the subject has sporadic ALS (sALS).

In an aspect, provided herein is a method of treating a disease or disorder associated with an increased level of oxidized phosphatidylcholine (OxPC) in a subject in need thereof, the method comprising administering to the subject a therapeutically effective amount of: (a) an antibody or antigen-binding fragment thereof that specifically binds to OxPC; or (b) a polynucleotide encoding an antibody or antigen-binding fragment thereof that specifically binds to OxPC.

In an embodiment, the disease or disorder is amyotrophic lateral sclerosis (ALS), Alzheimer's disease, motor neuron disease, Parkinson's disease, or frontotemporal lobar degeneration. In an embodiment, the disease or disorder is sporadic ALS (sALS).

In an embodiment, the rAAV is administered to the subject intravenously, intraperitoneally, subcutaneously, intramuscularly, intrathecally, or intradermally.

In an embodiment, the method neutralizes OxPC activity in the subject.

In an embodiment, the method prevents or reduces TDP-43 aggregates. In an embodiment, the method results in about a 1, 5, 10, 20, 30, 40, 50, 60, 70, 80, 90, or 100% reduction in TDP-43 aggregates in the subject.

In an embodiment, the method reduces the expression of one or more ALS related gene. In an embodiment, the one or more ALS related gene is apoE, COL4A1, CTSS, DAB2, TIMP1, or any combination thereof. In an embodiment, the method results in about a 1, 5, 10, 20, 30, 40, 50, 60, 70, 80, 90, or 100% reduction in expression of the one or more ALS related gene in the subject.

In an embodiment, the method increases the expression of one or more ALS related gene. In an embodiment, the one or more ALS related gene is C9orf72, GRM3, SYP, GRIN2B, CHRNA7, MYD88, ITPR2, GRN, VEGFA, FKBP5, or any combination thereof. In an embodiment, the method results in about a 1, 5, 10, 20, 30, 40, 50, 60, 70, 80, 90, or 100% increase in expression of the one or more ALS related gene in the subject.

In an embodiment, the method neutralizes OxPC mediated neurotoxicity. In an embodiment, the method increases electrical firing of a neuron. In an embodiment, the method increases neurite outgrowth on a neuron.

In an embodiment, the vector targets a cortical neuron, a spinal neuron, and/or an astrocyte.

In an embodiment, the subject is a human subject.

In an embodiment, the method further comprises administering an additional therapeutic agent.

In an aspect, provided herein is an antibody or antigen-binding fragment thereof that specifically binds to OxPC or a polynucleotide encoding an antibody or antigen-binding fragment thereof that specifically binds to OxPC for use in inhibiting TDP-43 aggregate-induced neurotoxicity in a subject in need thereof, wherein the treatment is performed according to any one of the methods disclosed herein.

In an aspect, provided herein is an antibody or antigen-binding fragment thereof that specifically binds to OxPC or a polynucleotide encoding an antibody or antigen-binding fragment thereof that specifically binds to OxPC for use in inhibiting oxidized phospholipid (OxPL)-induced neurotoxicity in a subject in need thereof, wherein the treatment is performed according to any one of the methods disclosed herein.

In an aspect, provided herein is an antibody or antigen-binding fragment thereof that specifically binds to OxPC or a polynucleotide encoding an antibody or antigen-binding fragment thereof that specifically binds to OxPC for use in preventing or reducing TDP-43 aggregates in a subject in need thereof, wherein the treatment is performed according to any one of the methods disclosed herein.

In an aspect, provided herein is an antibody or antigen-binding fragment thereof that specifically binds to OxPC or a polynucleotide encoding an antibody or antigen-binding fragment thereof that specifically binds to OxPC for use in the manufacture of a medicament for inhibiting TDP-43 aggregate-induced neurotoxicity in a subject in need thereof, wherein the treatment is performed according to any one of the methods disclosed herein.

In an aspect, provided herein is an antibody or antigen-binding fragment thereof that specifically binds to OxPC or a polynucleotide encoding an antibody or antigen-binding fragment thereof that specifically binds to OxPC for use in the manufacture of a medicament for inhibiting oxidized phospholipid (OxPL)-induced neurotoxicity in a subject in need thereof, wherein the treatment is performed according to any one of the methods disclosed herein.

In an aspect, provided herein is an antibody or antigen-binding fragment thereof that specifically binds to OxPC or a polynucleotide encoding an antibody or antigen-binding fragment thereof that specifically binds to OxPC for use in the manufacture of a medicament for preventing or reducing TDP-43 aggregates in a subject in need thereof, wherein the treatment is performed according to any one of the methods disclosed herein.

In an aspect, provided herein is a use of an antibody or antigen-binding fragment thereof that specifically binds to OxPC or a polynucleotide encoding an antibody or antigen-binding fragment thereof that specifically binds to OxPC for inhibiting TDP-43 aggregate-induced neurotoxicity in a subject in need thereof, wherein the treatment is performed according to any one of the methods disclosed herein.

In an aspect, provided herein is a use of an antibody or antigen-binding fragment thereof that specifically binds to OxPC or a polynucleotide encoding an antibody or antigen-binding fragment thereof that specifically binds to OxPC for inhibiting oxidized phospholipid (OxPL)-induced neurotoxicity in a subject in need thereof, wherein the treatment is performed according to any one of the methods disclosed herein.

In an aspect, provided herein is a use of an antibody or antigen-binding fragment thereof that specifically binds to OxPC or a polynucleotide encoding an antibody or antigen-binding fragment thereof that specifically binds to OxPC for preventing or reducing TDP-43 aggregates in a subject in need thereof, wherein the treatment is performed according to any one of the methods disclosed herein.

### EXAMPLES

### Example 1: OxPC Induces ALS-Like Transcriptome Signatures in Motor Neurons

Familial ALS (fALS) accounts for 5-10% of ALS diagnoses and cell lines harboring fALS mutations can be used to study ALS mechanisms in vitro. Therefore, two well-characterized ALS motor neuron cell lines (TDP-43^{M337V} and SOD1^{G93A}) were used to assess the OxPC profile and transcriptome in ALS, compared with healthy motor neurons treated with OxPC.

### a) OxPC Profile

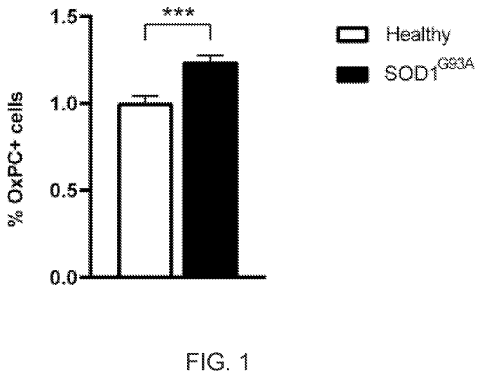

To assess the OxPC profile, the cells were washed with PBS, permeabilized for 15 minutes with 0.5% Saponin (47036-50G-F, Sigma), washed three times with PBS, and incubated with blocking solution (5% goat serum [31873, Thermo Fisher Scientific]) for 45 minutes at room temperature. The cells were then incubated with a mouse anti-phosphatidylcholine antibody (Absolute Antibody) diluted in blocking solution overnight at 4° C. or for 1 hour at room temperature. Following three washes in 1% goat serum, cells were incubated with the secondary antibody diluted in blocking solution in the dark for 1 hour at room temperature, then washed two times with PBS and incubated with Hoechst (1:10000 in PBS) in the dark for 10 minutes at room temperature. After one wash with PBS, the cells were stored at 4° C. until imaging. The results from this analysis showed an increase in the number of OxPC-positive cells in the SOD1^{G93A} ALS motor neurons, compared to healthy motor neurons (FIG. 1).

### b) Transcriptome Analysis

Next, transcriptome analysis of SOD1^{G93A} ALS motor neurons and healthy motor neurons was performed using NanoString Technology to identify the molecular mechanisms activated in the presence of OxPC.

One of the main OxPC species in rat, porcine, and human brain, I-palmitoyl-2-(9-oxo-nonanoyl)-sn-glycero-3-phosphocholine (PONPC) (870605P-1MG, Avanti Polar Lipids), was used as the OxPC treatment in this analysis. First, PONPC was dissolved in 100% ethanol (EtOH) and heated to 35° C. followed by sonication (37 kHz) for 30-60 seconds. The PONPC solution was diluted in complete motor neuron maintenance medium to 2× the final concentration. The motor neurons were treated with 25-100 μM PONPC and incubated for 24 hours or 24 hours followed by a 24-hour washout. 0.3% EtOH was used as a vehicle control. The motor neuron samples were prepared for NanoString analysis as described in the NanoString technologies gene expression hybridization protocol (Gene Expression CodeSet RNA Hybridization Protocol; MAN-10056-05). Two pre-designed NanoString Neuroscience panels were selected for gene expression analysis: (a) neuropathology and (b) neuroinflammation. The quality control and expression changes were analyzed with the nSolver 4.0 analysis software.

Of the 770 transcripts contained on each panel, exposure of healthy motor neurons to OxPC led to 174 (Neuropathology) and 161 (Neuroinflammation) differently expressed genes. OxPC exposure of SOD1^{G93A} ALS motor neurons led to differential expression of 137 genes (Neuroinflammation). Of all transcripts analyzed in healthy motor neurons, approximately 25% displayed a response to OxPC exposure. A similar profile was observed the same analysis was performed in SOD1^{G93A} ALS motor neurons. Gene Ontology (GO) analysis of differently expressed genes after OxPC treatment of healthy motor neurons revealed that the most represented pathways were: response to stimulus, programmed cell death, apoptosis, regulation of molecular functions, transcriptome remodeling, and protein phosphorylation. An approximate 50% overlap was found among differently expressed genes in both healthy and SOD1^{G93A} ALS motor neurons when exposed to OxPC. These results indicate that OxPC induces specific mechanisms and pathways in motor neurons that may be relevant in ALS pathology.

Transcriptome analysis of TDP-43^{M337V} and SOD1^{G93A} ALS motor neurons was performed to find common gene expression signatures between both cell lines. Similarities were observed in each panel between TDP-43^{M337V} and SOD1^{G93A} transcriptomes (NP=35.08%; NI=34.35%). Considerable transcriptome overlap was also found between healthy motor neurons exposed to OxPC and SOD1^{G93A} ALS motor neurons (NP=39.69%; NI=50.31%) and, to a lesser extent, between healthy motor neurons and TDP-43^{M337V} motor neurons (NP=28.48%; NI=50.31%). Of all differently expressed transcripts, 31.03% (NP) and 22.58% (NI) were common among the three motor neuronal cell lines (healthy, TDP-43^{M337V}, and SOD1^{G93A}).

Data mining analysis was performed using the transcriptomic data described above and two independent ALS transcriptome datasets: (1) ALSoD (Abel et al. 2012) and (2) postmortem spinal cord tissue from patients with sporadic ALS (D'Erchia et al. 2017). The analyses revealed that 20% of the transcripts that were previously related to ALS had altered expression following OxPC treatment of healthy motor neurons. These transcripts are listed in Table 3, along with a brief description of their function.

TABLE 3
OxPC-sensitive transcripts in motor neurons with a role in ALS. The Human Protein
Atlas (proteinatlas.org) was used to describe gene names and functions.

| Gene | Gene description | Function |
|---|---|---|
| ACTN1 | Actinin alpha 1 | Actin binding |
| APOE | Apolipoprotein E | Lipid metabolism, cholesterol metabolism, |
| | | lipid transport, steroid metabolism |
| ARHGAP44 | Rho GTPase activating | GTPase activation, exploratory dendritic |
| | protein 44 | filopodia |
| ATP2B3 | ATPase plasma membrane | Intracellular calcium homeostasis |
| | Ca2+ transporting 3 | |
| ATP8A2 | ATPase phospholipid | Lipid flipping, lipid transport |
| | transporting 8A2 | |
| ATXN2 | Ataxin 2 | Endocytosis, mTOR signals, ribosomal |
| | | translation, mitochondrial function |
| C9orf72 | C9orf72-SMCR8 complex | Autophagy, endosomal trafficking, endocytic |
| | subunit | transport |
| CABLES1 | Cdk5 and Abl enzyme | Neuronal outgrowth |
| | substrate 1 | |
| CACNA1B | Calcium voltage-gated | Calcium neuronal signaling |
| | channel subunit alpha1 B | |
| CADPS | Calcium dependent secretion | Vesicular exocytosis of neurotransmitters and |
| | activator | neuropeptides |
| CALB2 | Calbindin 2 | Calcium signaling |
| CHAT | Choline O-acetyltransferase | Biosynthesis of acetylcholine |
| CHMP2B | Charged multivesicular body | Endosomal transport, lysosomal degradation |
| | protein 2B | of membrane proteins, lysosomal enzymes, |
| | | and lipids |
| CHRNA7 | Cholinergic receptor nicotinic | Acetylcholine receptor, ligand-gated ion |
| | alpha 7 subunit | channel |
| CHRNB2 | Cholinergic receptor nicotinic | Acetylcholine receptor, ligand-gated ion |
| | beta 2 subunit | channel |
| CNTF | Ciliary neurotrophic factor | Survival factor, prevention of motor axons |
| | | degeneration |
| COL4A1 | Collagen type IV alpha 1 | Angiogenesis, extracellular matrix |
| | chain | homeostasis |
| CTSS | Cathepsin S | Protein degradation |
| DAB2 | DAB adaptor protein 2 | Apoptosis, Differentiation, Endocytosis, |
| | | Protein transport, Wnt signaling pathway |
| FKBP5 | FKBP prolyl isomerase 5 | Immunoregulation, protein folding and |
| | | trafficking |
| FRMPD4 | FERM and PDZ domain | Excitatory synaptic transmission |
| | containing 4 | |
| GRIN2A | Glutamate ionotropic | Calcium permeability, synaptic plasticity |
| | receptor NMDA type subunit | |
| | 2A | |
| GRIN2B | Glutamate ionotropic | Calcium permeability, synaptic plasticity |
| | receptor NMDA type subunit | |
| | 2B | |
| GRM3 | Glutamate metabotropic | G-protein coupled receptor for glutamate |
| | receptor 3 | |
| GRN | Granulin precursor | Lysosomal regulation, Inflammatory response |
| IL10RA | Interleukin 10 receptor | Inflammatory response |
| | subunit alpha | |
| IL13RA1 | Interleukin 13 receptor | Inflammatory response |
| | subunit alpha 1 | |
| ITPR2 | Inositol 1,4,5-trisphosphate | Calcium transport |
| | receptor type 2 | |
| MYD88 | MYD88 innate immune | Inflammatory response |
| | signal transduction adaptor | |
| RAB3C | RAB3C, member RAS | Protein transport |
| | oncogene family | |
| SLC12A5 | Solute carrier family 12 | Potassium-chloride cotransport |
| | member 5 | |
| SOD2 | Superoxide dismutase 2 | Oxidoreductase |
| SYP | Synaptophysin | Synaptic plasticity |
| TIMP1 | TIMP metallopeptidase | Metalloproteinase inhibitor, cell proliferation, |
| | inhibitor 1 | anti-apoptotic function |
| TNFRSF1A | TNF receptor superfamily | Inflammation, apoptosis |
| | member 1A | |
| UNC13A | Unc-13 homolog A | Neurotransmitter release |
| VIM | Vimentin | Cell shape maintenance, cytoskeletal |
| | | homeostasis, cytoplasm integrity |

### Example 2: OxPC Triggers Phenotypic Changes in ALS Motor Neurons, Including Disrupted Neuronal Network and TDP-43 Aggregation

Fully differentiated healthy and TDP-43^{M337V} ALS motor neurons were maturated in culture and exposed to PONPC (OxPC). Then, phenotypical changes that included neurite outgrowth, lipid peroxidation hallmarks, TDP-43 aggregation and phosphorylation, and apoE profile were assessed.

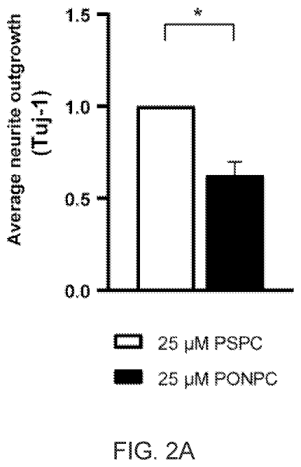

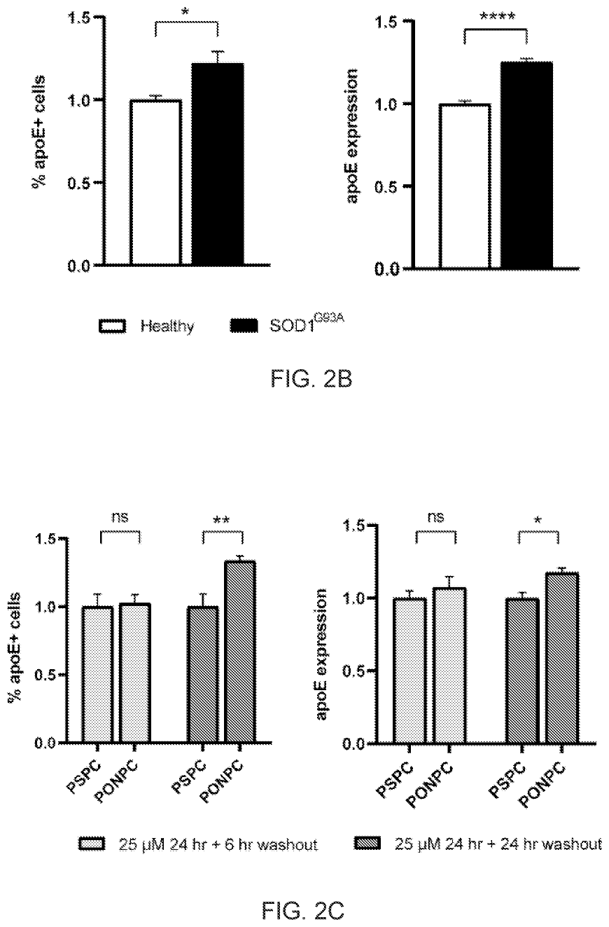

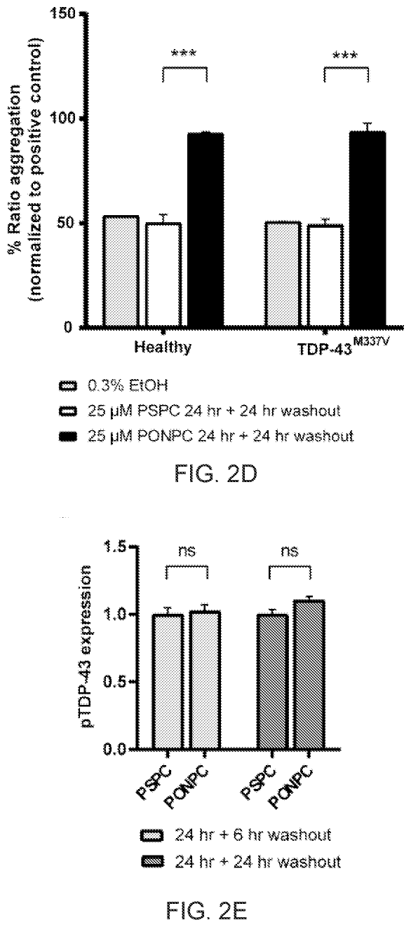

Healthy motor neurons and TDP-43^{M337V} ALS motor neurons were treated with 25 μM PONPC or 1-palmitoyl-2-stearoyl-sn-glycero-3-phosphocholine (PSPC) control for 24 hours. Average neurite outgrowth was quantified by running the neurite tracing protocol on CellReporterXpress Software, and percentage of positive cells and all cell average intensities were quantified by running the cell scoring protocol. Exposure of healthy motor neurons to OxPC (25 μM) led to a dramatic decrease in neurite outgrowth (FIG. 2A). Mitochondrial dysfunction is an important marker of oxidative stress. However, there were no differences on mitochondrial profile following OxPC exposure in either healthy motor neurons or TDP-43^{M337V} ALS motor neurons.

In the CNS, apoE participates in lipid redistribution in order to supply different cell types with cholesterol and phospholipids, which are required for their biological processes. When analyzing biofluids from ALS patients, preferential OxPL content was found on apoE in cerebrospinal fluid (CSF), but not in plasma. To further confirm the link between apoE, OxPC and ALS, apoE expression was evaluated in ALS motor neurons and in healthy motor neurons after OxPC treatment. The cells were washed with PBS and permeabilized with 0.1% Triton X-100 for 15 minutes at room temperature, washed three times with PBS, and incubated with blocking solution (3% BSA [422371X, VWR]) for 30 minutes at room temperature. Cells were incubated with mouse anti-apoE antibody (NovusBio) diluted in blocking solution overnight at 4° C., or for 1 hour at room temperature. The cells were washed three times with PBS, then incubated with the secondary antibody diluted in blocking solution in the dark for 1 hour at room temperature. Cells were washed two times with PBS and incubated with Hoechst (H3570, Invitrogen) diluted 1:10000 in PBS in the dark for 10 minutes at room temperature. Increased apoE expression was detected in SOD1^{G93A} ALS motor neurons (FIG. 2B) and in healthy motor neurons treated with OxPC (FIG. 2C).

TDP-43 aggregation and phosphorylation are important hallmarks in ALS pathology. Healthy motor neurons and TDP-43^{M337V} ALS motor neurons were treated with OxPC and analyzed for TDP-43 aggregation. The TDP-43 aggregates were analyzed with the HTRF kit (TDP-43 aggregation kit, cisbio) according to the manufacturer's instructions. Increased TDP-43 aggregation was observed 24 hours post-OxPC treatment of both healthy and ALS motor neurons (FIG. 2D). Phospo-TDP-43 (pTDP-43) was analyzed by IHC with a rabbit anti-pTDP-43 antibody (Proteintech), according to the method described for ApoE detection above. The results showed a trend for increased pTDP-43 expression in healthy motor neurons treated with OxPC under similar conditions (FIG. 2E), though this effect was not statistically significant.

Next, the functional consequences of OxPC were evaluated by assessing the electrical activity profile of healthy and TDP-43^{M337V} ALS motor neurons exposed to OxPC. Multielectrode array (MEA) activity was monitored on days 7, 16, 20, 21 and 22 post-cell seeding with Maestro Pro (Axion Biosystems). Electrical activity was recorded according to the following settings: Neural Spikes detection (configuration), 12.5 kHz (Sampling frequency), and 200 Hz-3k Hz (Filter). Data analysis included the following settings: 0-180 sec (analysis start and end), 5 spks/min (active electrode criterion), 11 kOhms (Minimum Covered Resistance), Remove Coincident Artifacts Electrode Burst Settings-Algorithm Poisson Surprise, 5 (Min surprise), Network Burst Settings-Algorithm Envelope, 1.25 (Threshold Factor), 100 ms (Min IBI), 75% (Burst inclusion): 35% (Min of electrodes). Exclusion criteria were applied to wells that showed poor cell attachment/survival, less than four active electrodes and or weighted mean firing rate (wMFR) below 1.5 Hz. The assay was performed with four biological replicates. Prior to OxPC treatment on day 21, ALS motor neurons consistently displayed lower MEA activity at different time points and metrics analyzed in comparison to healthy motor neurons. A slight decrease on wMFR was observed on healthy, but not on ALS motor neurons, following OxPC treatment. OxPC exposure caused decreased burst activity on both healthy motor neurons and ALS motor neurons,

### Example 3: Vectorized Anti-OxPC scFv is Expressed in the CNS and Targets ALS-Relevant Cell Types

The limited permeability of the blood-brain barrier (BBB) constitutes the major obstacle in the use of antibody-based therapies for CNS diseases. To overcome the BBB obstacle, a vectorized anti-OxPC scFv (E06) was developed as an antibody treatment strategy to neutralize OxPC in CNS cell types (e.g., motor neurons, astrocytes). AAV5.2 was chosen as the AAV capsid for this strategy because it is known to target cells within the CNS. Briefly, AAV5.2 carrying the sequences for vectorized mouse E06-scFv (AAV5.2-CBh-mE06) and humanized E06-scFv (AAV5.2-CBh-hE06), and a control AAV5.2-CAG-GFP were produced in a baculovirus expression system. The resulting recombinant AA Vs are referred to herein as “AAV5.2-mE06”, “AAV5.2-hE06”, and “AAV5.2-GFP”.

To test the transduction efficiency of the recombinant AAVs, Human iPSC-derived neurons and astrocytes were transduced with AAV5.2-mE06 or AAV5.2-GFP at a MOI of 10⁵, 10⁶, and 10⁷ in culturing media 7-10 days post-seeding. Half of the medium was removed from the wells before adding the AAV mixture. The same volume of fresh media was added to the cells 4 hours post-transduction.

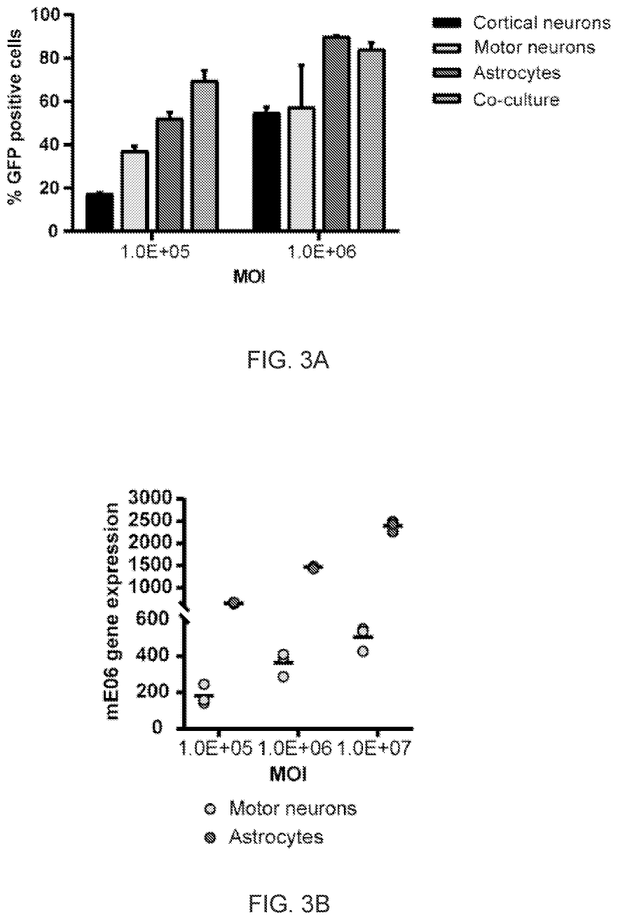

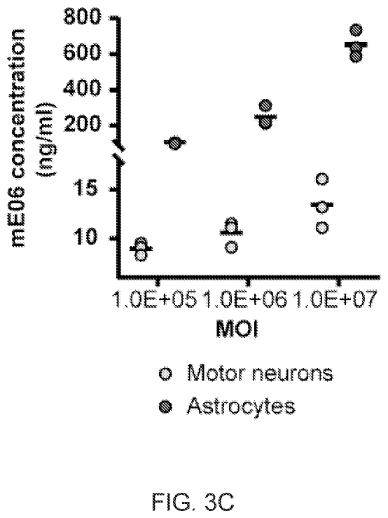

AAV5.2-GFP was first assessed to determine the ability of AAV5.2 to transduce CNS cell types. ALS-relevant cell types like astrocytes and co-cultures of astrocytes and neurons displayed high transduction rates (60-80%, FIG. 3A). In motor neurons, around 40-60% of cells were successfully transduced by AAV5.2-GFP.

Transduction and expression were correlated in a dose dependent manner as measured by mE06 mRNA (FIG. 3B) and protein (FIG. 3C). In summary, AAV5.2-mE06 is highly efficient in transducing and expressing mE06 in motor neurons and astrocytes.

### Example 4: Vectorized Anti-OxPC scFv Normalizes OxPC-Induced Pathways and Expression Changes

The potential of AAV5.2-mE06 in neutralizing the effects of OxPC was evaluated by performing the NanoString transcriptome assay (as described in Example 1 above) in (i) healthy motor neurons exposed to OxPC and (ii) SOD1^{G93A} ALS motor neurons, transduced with AAV5.2-mE06. Transcriptome analysis showed that transduction with AAV5.2-mE06 was associated with important biological processes including regulation of cell death, apoptosis, protein metabolism, molecular function, and response to endogenous stimulus. In contrast, processes such as aging and protein phosphorylation were solely enriched in SOD1^{G93A} ALS motor neurons transduced with AAV5.2-mE06.

In most cases, treatment with AAV5.2-mE06 reversed the transcriptome changes caused by OxPC treatment in healthy neurons. Approximately 40% of all OxPC-sensitive transcripts showed some improvement (>0.1-fold change) following transduction with AAV5.2-mE06, see Table 4 below. Similar effects were observed after AAV5.2-mE06 transduction of SOD1^{G93A} ALS motor neurons that were not treated with OxPC (Table 2).

TABLE 4
Selected genes that have altered expression ALS motor neurons
(SOD1^{G93A}) or in OxPC-treated healthy motor neurons.

| | Healthy cells | ALS motor neurons |
|---|---|---|
| | treated with OxPC | (SOD1^{G93A}) |
| Genes upregulated, and | apoE, COL4A1, CTSS, | SERPINF1, apoE, |
| downregulated following | DAB2, TIMP1 | LMNB1 |
| transduction with AAV5.2-mE06 | | |
| Genes downregulated by OxPC | C9orf72, GRM3, SYP, | CHRNA7, GABRG2, |
| treatment, and upregulated | GRIN2B, CHRNA7, | CTSS, GAD1, |
| following transduction with | MYD88, ITPR2, GRN, | TIMP1, PTGER3 |
| AAV5.2-mE06 | VEGFA, FKBP5, | |

### Example 5: Vectorized Anti-OxPC scFv Protects Against OxPC-Mediated Neurotoxicity

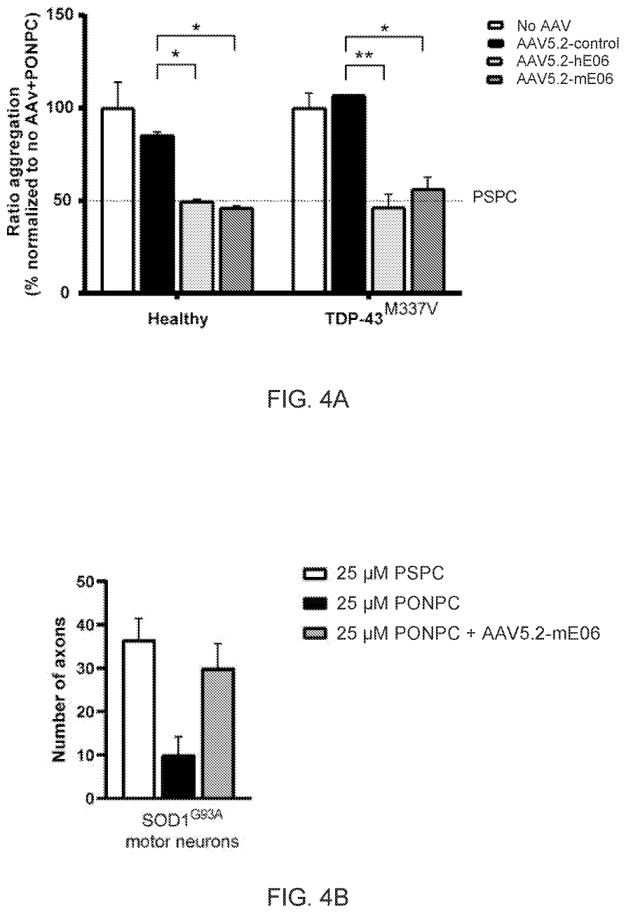

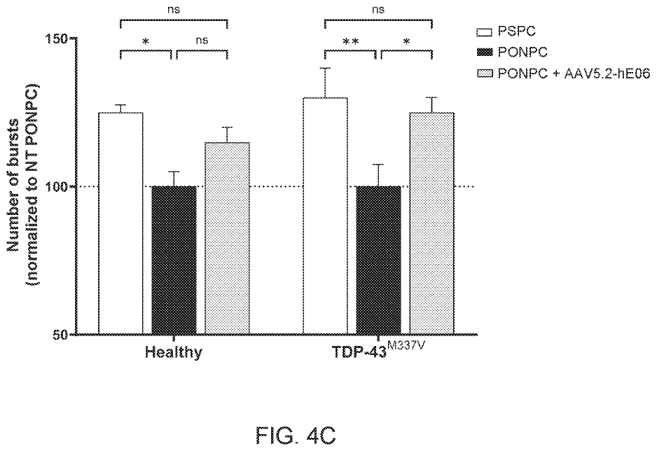

The ability of OxPC to induce TDP-43 aggregation in both healthy and ALS motor neurons was described above. It was evaluated whether this phenotype could be prevented using AAV5.2-mE06 or AAV5.2-hE06, transduced prior to treatment. Notably, both AAV5.2-mE06 and AAV5.2-hE06 were able to completely abrogate the TDP-43 aggregation caused by OxPC exposure (FIG. 4A).

Axonal degeneration is an important mechanism in motor neuron disease. Accordingly, the effect of OxPC toxicity on neuronal projections and axonal health was evaluated in SOD1^{G93A} ALS motor neurons. These neurons were used because of the established axonal impairments associated with this genotype. Using an OMEGA-NMJ chip (eNUVIO), a marked decrease (≈73%) was observed in the number of projecting axons in SOD1^{G93A} ALS motor neurons treated with OxPC (FIG. 4B). AAV5.2-mE06 transduction prior to OxPC treatment practically reverted this toxicity to OxPC-untreated levels (≈80%).

Next, the ability of AAV5.2-hE06 to prevent the effects of OxPC on healthy and TDP-43^{M337V} ALS motor neurons was evaluated by assessing electrical activity profile. Briefly, 125,000 healthy and 25,000 TDP-43^{M337V} ALS motor neurons/astrocytes were seeded in a co-culture setting on a Cyto View 96-well plate (M768-tMEA-96B, Axion Biosystems) and transduced with AAV5.2-hE06 prior to treatment with OxPC at MOI 10⁶. Assessment of Multielectrode array (MEA) activity was performed as in Example 2. The assay was performed with four biological replicates. A decrease in the number of bursts was observed in cells receiving only OxPC treatment while this decrease was prevented in cells that were transduced with AAV5.2-hE06 prior to OxPC treatment (FIG. 4C).

### Example 6: Vectorized Anti-OxPC scFv Protects Against Motor Deficits and Transmission of ALS Pathology in an In Vivo Mouse Model

Prior studies have reported neurotoxic properties of CSF derived from ALS patient and demonstrated transfer of disease pathology to animals injected with CSF derived from ALS patients (Wong et al., Brain Commun. 2022 Aug. 22; 4(4):fcac207. doi: 10.1093). The ability of AAV5.2-mE06 to prevent transmission of ALS pathology in mice injected with CSF from ALS patients (sALS-CSF mouse model) was investigated. The study was carried out at Tisch MS Research Center of New York (Tisch Center). In brief, CSF from sporadic ALS patients and control CSF were obtained from the CSF bank at the Tisch Center. Institutional Review Board approval and informed consent according to the Declaration of Helsinki was granted prior to CSF collection. Samples were collected with sterile techniques either by lumbar puncture or access port aspiration of surgically implanted pumps. CSF samples were centrifuged at 200×g for 15 min to remove cells, confirmed to be free of red blood cell contamination by microscopy, then stored in aliquots at −80° C.

Adult female C57BL/6J mice (aged 8-12 weeks) purchased from The Jackson Laboratory (Bar Harbor, ME) were used in all in vivo experiments. All procedures were approved by the Institutional Animal Care and Use Committee at Mispro Biotech Services (New York). Prior to surgery, mice were anaesthetized with a ketamine (110 mg/kg) and xylazine (10 mg/kg) cocktail and received subcutaneous injections of 0.1 mg/kg buprenorphine, 2.5 mg/kg baytril and 1 ml 0.9% saline. Laminectomies at cervical levels 4 (C4) and 5 (C5) were performed to expose the underlying spinal cord. A 32-gauge Hamilton syringe was inserted underneath the dura mater and 5 μL of saline/AAV5.2-mE06 or 3 μL of saline/CSF as control were slowly injected into the subarachnoid space. AAV5.2-mE06 was injected intrathecally in 8-week old mice, at a dose of 4.3e¹¹ genome copies (gc)/mouse followed by saline/CSF injection four weeks later (12-weeks old mice). A minimum of three mice were injected per individual patient CSF sample. Mice were assigned to different treatment groups in a randomized manner.

### a) Motor Deficit Score Testing

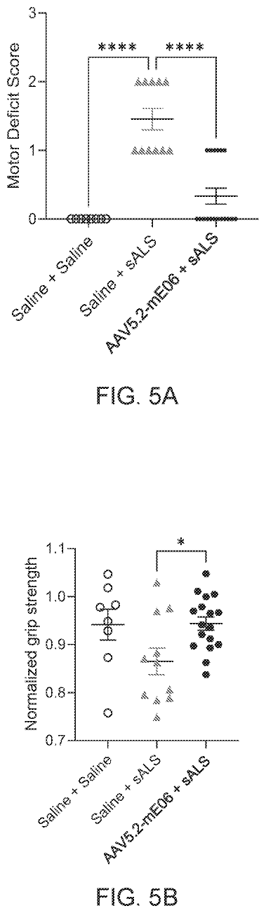

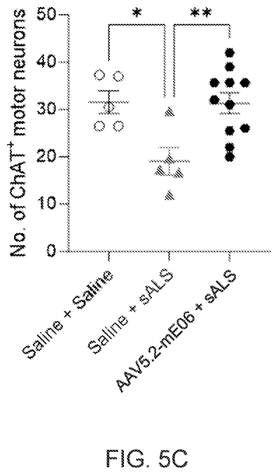

Following intrathecal delivery of CSF, all mice underwent motor testing at 1 day post-injection (DPI). Forelimb reaching, gripping and tail flaccidity were evaluated on a 3-point scale. Mice were held by their tails above their cage bars and allowed to reach out and grip the bars for five trials. Mice displaying no motor deficits were given a score of 0. Any deficit in either reaching or gripping were each given a score of 1. Specifically, an inaccurate reach was considered to be a reaching deficit, and weakness in grip strength or clenched forepaws were scored as gripping deficits. Tail flaccidity was also given a score of 1. All motor testing was performed blinded with respect to treatment groups. An increase in motor deficits was observed in mice treated with saline prior to the injection with sALS-CSF, while mice pretreated with AAV5.2-mE06 developed less motor deficits (FIG. 5A).

### b) Grip Strength Testing

Mice were habituated to the grip strength meter (TSE systems) for 3 days prior to surgery. Each mouse was given 1 min to explore the grip strength meter, then held by their tails and allowed to grip the bar with both forelimbs for five consecutive trials. After a 30 s rest period, the mouse was given another five trials to grip and then returned to their home cage. Baseline grip strength force was measured at 1 day prior to surgery and again at 1 DPI. The mean grip strength force was calculated from five trials. Normalized grip strength values were calculated by dividing mean grip strength force on post-injection testing day by mean baseline grip strength force. A decrease in grip strength was observed in mice treated with saline prior to the injection with sALS-CSF, while this was prevented in mice pretreated with AAV5.2-mE06 (FIG. 5B).

### c) Motor Neuron Histology

Tissue harvesting: Mice were sacrificed using an overdose of ketamine (300 mg/kg) and xylazine (30 mg/kg) and were perfused transcardially with phosphate buffered saline (PBS) followed by 4% paraformaldehyde in 0.1 M PBS, pH 7.4. Spinal cords and brains were dissected out, postfixed in 4% paraformaldehyde overnight and then placed in 30% sucrose overnight for cryoprotection.

Cervical spinal cords were cut 0.5 cm rostral and 0.5 cm caudal to the injection site, and 1 cm of thoracic spinal cords were also collected. The 1 cm segments were then embedded and frozen in Tissue Tek® (VWR International, PA). Spinal cords were sectioned sagittally at 20 μm thickness using a cryostat (Leica) and then slide-mounted onto Histobond® slides (VWR International, PA). The anatomical orientation of tissue sections, as well as the order and position in which they were mounted onto the slides were kept consistent to facilitate unbiased histological comparisons, as described in further detail below. Brains were sectioned coronally at 30 μm and free-floating sections were stored in 0.01% sodium azide in PBS.

Immunofluorescence staining: Immunostaining was performed on the series of spinal cord sections at 100 μm intervals throughout the entire cervical spinal cord or thoracic spinal cord. Slides with spinal cord sections or cells, or free-floating brain sections, were washed three times in 0.1% triton X-100 in PBS (PBS/T), then incubated in 10% normal goat serum (NGS) or normal donkey serum (NDS) in PBS/T for 1 h at room temperature. Primary antibody goat anti-ChAT (Millipore, 1:100) was diluted in 10% NGS or NDS in PBS/T and incubation occurred overnight at 4° C. After incubation, slides or sections were rinsed three times in PBS and incubated in a 1:750 dilution of the appropriate Alexa-Fluor secondary antibodies (Invitrogen) in 10% NGS or NDS in PBS/T for 1.5 h at room temperature. Slides or sections were rinsed three times in PBS and then counterstained with 1:2500 DAPI in PBS (Invitrogen) for 5 min. After two final washes in PBS, free-floating brain sections were mounted onto slides, and slides were mounted using Fluoromount (Sigma).

Histological analyses: Images were captured at 20× magnification using a Zeiss Axio Imager. Acquisition parameters and exposure times were kept consistent for each antibody stain. To ensure unbiased comparisons between experimental groups, spinal cord images were captured from similar tissue section numbers on the slides and matching anatomical regions were verified by experimenters. The number of motor neurons and immunostaining intensities were quantified using ImageJ software. For cell counts, three images were quantified per mouse to calculate the mean motor neuron number. Fluorescence intensities were measured as mean grey values in regions of interest. Both imaging and quantification were performed by experimenters blinded for treatment groups. A decrease in the number of motor neurons was observed in mice treated with saline prior to the injection with sALS-CSF while this was prevented in mice that were pretreated with AAV5.2-mE06 (FIG. 5C).

### Example 7: Vectorized Anti-OxPC scFv Normalizes OxPC Concentration in SOD1^{G93A} ALS Mouse Model

The ability of AAV5.2-mE06 to normalize levels of various OxPC species in a mouse model of ALS pathology was investigated.

SOD1^{G93A} transgenic (TG) and wild type (WT) mice were intrathecally administered either PBS/0.001% Pluronic or 1×10¹² gc/mouse AAV5.2-mE06 at age 45 days. A subset from each dose group was terminated and subjected to sample collection at ages 70 days and 90 days. The remaining mice per dose group were terminated at a humane end point criterion (survival), or when reaching age 160 days. Plasma was collected from 24 PBS-treated SOD1^{G93A} mice and 15 AAV5.2-mE06-treated SOD1^{G93A} mice and used for OxPC analysis.

To measure OxPC species, lipids were extracted from 50 μL mouse plasma according to the Folch method. The supernatants were evaporated to dryness and resolved with methanol. The resolved samples were analyzed using an Agilent 1290 HPLC system with binary pump, multisampler and column thermostat with a Zorbax Eclipse plus C-18, 50×2.1 mm, 1.8 μm, 40° C. column using a gradient solvent system consisting of mobile phase A: acetonitrile/water 60:40, 5 mM NH4Ac, 0.05% formic acid and mobile phase B: 2-propanol/acetonitrile 90:10, 5 mM NH4Ac, 0.05% formic acid. The gradient consisted of 32-97% B in 21 min, stop time after 27 min. The flow rate was set at 0.4 mL/min, the injection volume was 5 μL. The HPLC was coupled with an Agilent 6470 Triplequad mass spectrometer (Agilent Technologies, Santa Clara, USA) with electrospray ionization source parameters optimized. Analysis was performed with Multiple Reaction Monitoring according to Solati et al., Front. Med. 1; 8:716944 (2021), doi: 10.3389/fmed.2021.716944. The identification of the analytes was performed by the characteristic mass transition and retention time. For the quantification, external POVPC and SOVPC standards (Cayman Chemical, Ann Arbor, MI) were used and concentrations were expressed in ng/ml.

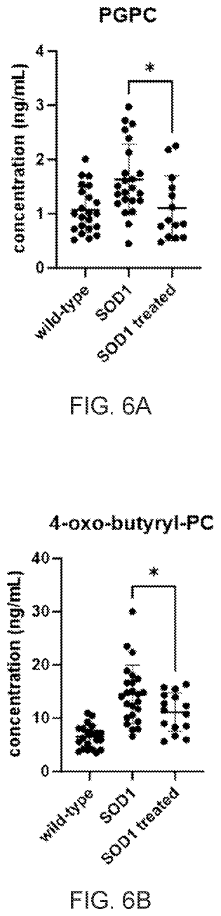

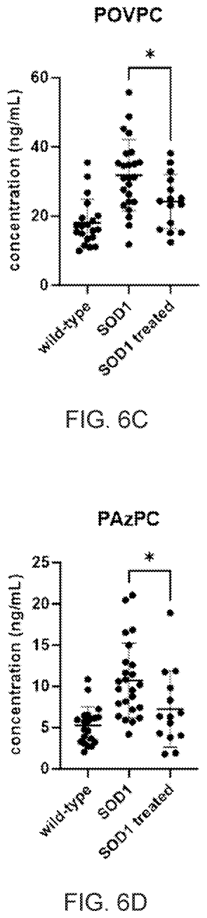

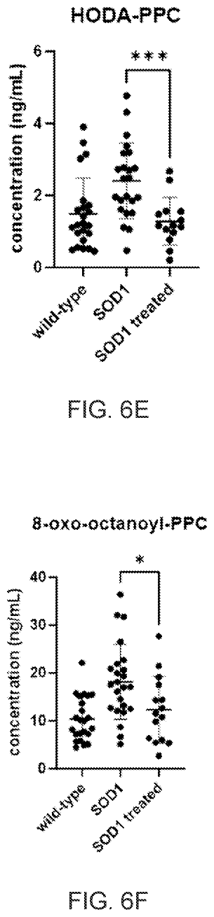

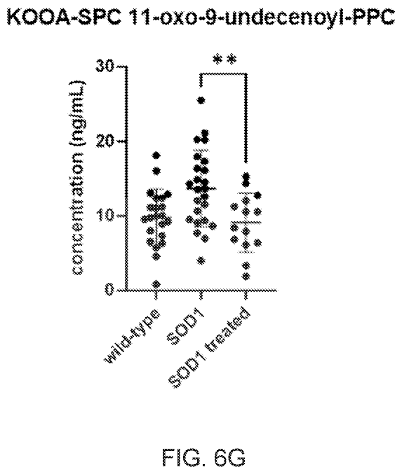

Out of the 22 different OxPC species previously identified in Solati et al. (supra), 19 were detected in the WT and TG SOD1^{G93A} mice. Seven of the measured OxPC species showed normalized OxPC concentrations upon treatment of SOD1^{G93A} mice with AAV5.2-mE06 (FIGS. 6A-6G).

### CONCLUSION

These results indicate that upon binding to OxPC, the anti-OxPC scFv neutralizes the neurotoxic and neuroinflammatory mechanisms of OxPC, as demonstrated by the prevention of TDP-43 aggregates and OxPC-induced neurotoxicity, as well as altered gene expression patterns and recovered in vivo OxPC levels in an ALS mouse model. Thus, an anti-OxPC scFv, particularly a vectorized anti-OxPC scFv, is a potential strategy for treating ALS.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1-54. (canceled)

55. A method of inhibiting oxidized phospholipid (OxPL)-induced neurotoxicity in a subject in need thereof, the method comprising administering to the subject a therapeutically effective amount of:
(a) an antibody or antigen-binding fragment thereof that specifically binds to oxidized phosphatidylcholine (OxPC); or
(b) a polynucleotide encoding an antibody or antigen-binding fragment thereof that specifically binds to OxPC.

56. The method of claim 55, wherein the subject has amyotrophic lateral sclerosis (ALS), Alzheimer's disease, motor neuron disease, Parkinson's disease, or frontotemporal lobar degeneration, or, wherein the subject has sporadic ALS (sALS).

57. The method of claim 55, wherein the antibody or antigen-binding fragment comprises a heavy chain variable region comprising complementarity determining regions CDRH1, CDRH2, and CDRH3 of the heavy chain variable region amino acid sequence set forth in SEQ ID NO: 7, and a light chain variable region comprising complementarity determining regions CDRL1, CDRL2, and CDRL3 of the light chain variable region amino acid sequence set forth in SEQ ID NO: 8.

58. The method of claim 55 wherein the antibody or antigen-binding fragment thereof comprises a heavy chain variable region comprising an amino acid sequence with at least 80% identity to SEQ ID NO: 7 and/or wherein the antibody or antigen-binding fragment thereof comprises a light chain variable region comprising an amino acid sequence with at least 80% identity to SEQ ID NO: 8.

59. The method of claim 58, wherein the antibody or antigen-binding fragment thereof is a humanized antibody.

60. The method of claim 57, wherein the antibody or antigen-binding fragment thereof is a single chain variable fragment (scFv), optionally, wherein the scFv comprises a peptide linker between the heavy chain variable region and the light chain variable region, such as the peptide linker with the amino acid sequence of GGGGSGGGGSGGGGS (SEQ ID NO: 9).

61. The method of claim 55, wherein the method neutralizes OxPC in the subject.

62. The method of claim 55, wherein the method prevents or reduces TDP-43 aggregates.

63. The method of claim 55, wherein the method reduces the expression of one or more ALS-related gene, such as one or more of ALS-related gene selected from apoE, COL4A1, CTSS, DAB2, and TIMP1, or any combination thereof.

64. The method of claim 55, wherein the method increases the expression of one or more ALS-related gene, such as one or more ALS-related gene selected from C9orf72, GRM3, SYP, GRIN2B, CHRNA7, MYD88, ITPR2, GRN, VEGFA, and FKBP5, or any combination thereof.

65. The method of claim 55, wherein the method:
neutralizes OxPC mediated neurotoxicity; or,
increases electrical firing of a neuron; or
increases neurite outgrowth on a neuron;

66. The method of claim 55, wherein the subject is a human subject.

67. The method of claim 55, wherein the antibody or the polynucleotide is administered to the subject intravenously, intraperitoneally, subcutaneously, intramuscularly, intrathecally, or intradermally.

68. The method of claim 55, wherein the polynucleotide is comprised within a vector, such as a viral vector.

69. The method of claim 68, wherein the vector targets a cortical neuron, a spinal neuron, and/or an astrocyte.

70. The method of claim 68, wherein the vector is a viral vector selected from the group consisting of adeno-associated virus (AAV), adenovirus, retrovirus, orthomyxovirus, paramyxovirus, papovavirus, picornavirus, lentivirus, herpes simplex virus, vaccinia virus, pox virus, and alphavirus.

71. The method of claim 68, wherein the vector is comprised within a recombinant AAV (rAAV), wherein the rAAV comprises an AAV capsid comprising an AAV capsid protein; and a rAAV genome.

72. The method of claim 71, wherein the capsid protein comprises an amino acid sequence that has at least 95% identity to SEQ ID NO: 10 or 11.

73. The method of claim 71, wherein the rAAV genome further comprises a CBh promoter comprising the nucleic acid sequence set forth in SEQ ID NO: 12.

74. The method claim 71, wherein the rAAV genome further comprises an SV40 polyA tail comprising the nucleic acid sequence set forth in SEQ ID NO: 13.

75. The method of claim 71, wherein the rAAV is administered to the subject intravenously, intraperitoneally, subcutaneously, intramuscularly, intrathecally, or intradermally.

76. A method of inhibiting TDP-43 aggregate-induced neurotoxicity in a subject in need thereof, the method comprising administering to the subject a therapeutically effective amount of:
(a) an antibody or antigen-binding fragment thereof that specifically binds to OxPC; or
(b) a polynucleotide encoding an antibody or antigen-binding fragment thereof that specifically binds to OxPC.

77. A method of preventing or reducing TDP-43 aggregates in a subject suffering from a condition associated with TDP-43 aggregate formation, the method comprising administering to the subject a therapeutically effective amount of:
(a) an antibody or antigen-binding fragment thereof that specifically binds to OxPC; or
(b) a polynucleotide encoding an antibody or antigen-binding fragment thereof that specifically binds to OxPC.
